(19) United States
(12) Patent Application Publication
(10) Pub. No.: US 2025/0295612 A1
(43) Pub. Date: Sep. 25, 2025

(54) NOVEL AMYLOID-BETA AGGREGATE DEGRADER AND BRAIN-TARGETING DRUG DELIVERY SYSTEM USING SAME

(71) Applicant: UNIVERSITY-INDUSTRY COOPERATION GROUP OF KYUNG HEE UNIVERSITY, Yongin-si (KR)

(72) Inventors: Dokyoung KIM, Seoul (KR); Jaehoon KIM, Seoul (KR); Hyeji UM, Namyangju-si (KR)

(73) Assignee: UNIVERSITY-INDUSTRY COOPERATION GROUP OF KYUNG HEE UNIVERSITY, Yongin-si (KR)

(21) Appl. No.: 19/232,251

(22) Filed: Jun. 9, 2025

Related U.S. Application Data
(63) Continuation of application No. PCT/KR2023/020069, filed on Dec. 7, 2023.

(30) Foreign Application Priority Data
Dec. 9, 2022 (KR) 10-2022-0171770
Mar. 8, 2023 (KR) 10-2023-0030332

(51) Int. Cl. A61K 31/145; A61K 9/51; A61K 47/69; A61P 25/28; C07C 309/47

(52) U.S. Cl. CPC A61K 31/145; A61K 9/5115; A61K 47/6929; A61P 25/28; C07C 309/47

(57) ABSTRACT

The present invention comprises: a novel molecule capable of disaggregating amyloid-beta plaques; and a brain-targeting amyloid-beta plaque disaggregation nano platform loaded with the molecule. An amyloid-beta plaque disaggregating agent developed according to the present invention exhibits high amyloid-beta plaque disaggregation efficacy, and the brain-targeting amyloid-beta plaque disaggregation nano platform shows high potential in the medical field on the basis of the effects of effectively targeting the brain and disaggregating amyloid-beta plaques present in the brain.

### CROSS REFERENCE TO RELATED APPLICATIONS

This application is a Bypass Continuation Application of International Application No. PCT/KR2023/020069 filed on Dec. 7, 2023, claiming priority based on Korean Patent Application No. 10-2022-0171770 filed on Dec. 9, 2022 and Korean Patent Application No. 10-2023-0030332 filed on Mar. 8, 2023, the entire disclosures of which are incorporated herein by reference.

### TECHNICAL FIELD

The present disclosure relates to a method for brain-targeting drug delivery of ANA using the efficacy of 6-amino-2-naphthalenesulfonic acid (ANA) in disaggregating amyloid-beta plaques and porous silicon nanoparticles.

### BACKGROUND ART

As the aging society worsens worldwide, the incidence of degenerative brain diseases such as Alzheimer's disease, Parkinson's disease, and Huntington's disease is also increasing. Among them, Alzheimer's disease is rapidly increasing in prevalence every year, and is known to account for 50 to 60% of all dementia. Since Alzheimer's disease was first reported in 1906, many studies have been conducted in the fields of etiology, lesions, symptoms, and treatment, but the exact cause and treatment thereof have not yet been clearly identified.

Among hypotheses presumed as the cause of Alzheimer's disease, amyloid hypothesis is currently closest to the cause of Alzheimer's disease. Amyloid-beta monomers, which may exist in all parts of the body including the brain, may form amyloid-beta plaques that cause serious toxicity in the brain of Alzheimer's disease patients. Currently, many treatment methods, such as a BACE1 (β-secretase enzyme) inhibitor, anti-amyloid-beta antibodies, and the like, have been developed based on the amyloid hypothesis, but the development of a treatment method of directly disaggregating and removing amyloid-beta plaques in the brain is a problem to be prioritized most of all.

Based on the results of showing that sulfonic acid may be involved in amyloid-beta plaques in the previous studies, the present inventors constructed a chemical library consisting of 20 sulfonic acid-based materials, and selected 6-amino-2-naphthalenesulfonic acid (ANA) as an optimal amyloid-beta plaque disaggregating agent through library-based screening. The ANA showed high amyloid-beta plaque disaggregation efficacy even at low concentrations in vitro, and an amyloid-beta plaque disaggregation nano platform (APDN) loaded with ANA and conjugated with a brain-targeting material in porous silicon nanoparticles demonstrated high amyloid-beta plaque disaggregation efficacy along with high biostability not only in vitro but also in an animal model induced by amyloid-beta plaques.

### DISCLOSURE

### Technical Problem

An object of the present disclosure is to discover a novel amyloid-beta plaque disaggregating agent, and further to provide a novel brain-targeting drug delivery method using the same.

### Technical Solution

In order to achieve the object,
the present disclosure provides a compound represented by the following Chemical

Formula 1:

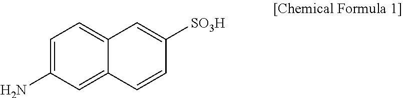 [SMILES: Nc1ccc2cc(S(=O)(=O)O)ccc2c1]

In one embodiment, the compound is 6-amino-2-naphthalenesulfonic acid (ANA) and is an agent for disaggregating amyloid-beta plaques.

In one embodiment, the compound is an agent that disaggregates amyloid-beta plaques in a concentration-dependent manner.

In one embodiment, the compound may exhibit high amyloid-beta plaque disaggregation efficacy under conditions of pH 3 to 5, 7, and 9.

In one embodiment, the compound may exhibit high amyloid-beta plaque disaggregation efficacy under conditions of not only in vitro but also in vivo.

In one embodiment, the compound may exhibit efficacy in improving memory loss under an in vivo condition.

In one embodiment, the compound may be developed as a brain-targeting amyloid-beta plaque disaggregation nano platform (APDN).

Therefore, the present disclosure provides a nano platform including a compound represented by Chemical Formula 1, a hydrate thereof, a solvate thereof, or a pharmaceutically acceptable salt thereof.

In the present disclosure, unless otherwise specified, the “nano platform” and similar terms should be given the broadest meaning and include particles, materials and compositions having a volume shape having at least one dimension from about 1 nanometer (nm) to about 500 nm. Preferably, in embodiments, such a volume shape may have a largest cross-section of about 100 nm to about 300 nm. More preferably, the volume shape may have a size of about 200 nm. In addition, the nano platform may include a backbone material, such as a cage, support or matrix material, and one or more additives bound with the backbone, such as agents, moieties, compositions, biologics, and molecules. In general, the backbone material may be nanoparticles.

In addition, any type of nano carrier may be used in the nano platform according to the present disclosure.

Further, the present disclosure provides a method for manufacturing a nano platform including: preparing porous silicon nanoparticles; loading a compound represented by Chemical Formula 1, a hydrate thereof, a solvate thereof, or a pharmaceutically acceptable salt thereof onto the porous silicon nanoparticles; sealing the surface of the loaded porous silicon nanoparticles with calcium chloride; and surface-modifying the coated porous silicon nanoparticles with biotin-polyethyleneglycerol (biotin-PEG).

Further, the present disclosure provides a composition for disaggregating amyloid-beta plaques, including the compound represented by Chemical Formula 1, a hydrate thereof, a solvate thereof, or a pharmaceutically acceptable salt thereof.

Further, the present disclosure provides a composition for disaggregating amyloid-beta plaques, including the nano platform according to the present disclosure.

Further, the present disclosure provides a pharmaceutical composition for preventing or treating a disease caused by amyloid-beta plaques, including the compound represented by Chemical Formula 1, a hydrate thereof, a solvate thereof, or a pharmaceutically acceptable salt thereof.

Further, the present disclosure provides a pharmaceutical composition for preventing or treating a disease caused by amyloid-beta plaques, including the nano platform according to the present disclosure.

Further, the present disclosure provides a pharmaceutical composition for preventing or improving memory loss, including the compound represented by Chemical Formula 1, a hydrate thereof, a solvate thereof, or a pharmaceutically acceptable salt thereof.

Further, the present disclosure provides a pharmaceutical composition for preventing or improving memory loss, including the nano platform according to the present disclosure.

Further, the present disclosure provides a method for disaggregating amyloid-beta plaques, including administering to a subject the compound represented by Chemical Formula 1, a hydrate thereof, a solvate thereof, or a pharmaceutically acceptable salt thereof.

Further, the present disclosure provides a method for disaggregating amyloid-beta plaques, including administering to a subject the nano platform according to the present disclosure.

Further, the present disclosure provides a method for preventing or treating a disease caused by amyloid-beta plaques, including administering to a subject the compound represented by Chemical Formula 1, a hydrate thereof, a solvate thereof, or a pharmaceutically acceptable salt thereof.

Further, the present disclosure provides a method for preventing or treating a disease caused by amyloid-beta plaques, including administering to a subject the nano platform according to the present disclosure.

Further, the present disclosure provides a method for preventing or improving memory loss, including administering to a subject the compound represented by Chemical Formula 1, a hydrate thereof, a solvate thereof, or a pharmaceutically acceptable salt thereof.

Further, the present disclosure provides a method for preventing or improving memory loss, including administering to a subject the nano platform according to the present disclosure.

### Advantageous Effects

The compound of the present disclosure, 6-amino-2-naphthalenesulfonic acid (ANA), can disaggregate amyloid-beta plaques with high efficacy not only in vitro but also in vivo, and the brain-targeting amyloid-beta plaque disaggregation nano platform using the same has advantages of being able to directly deliver drugs to the brain and disaggregate amyloid-beta plaques in the brain to eliminate an actual pathogenesis of Alzheimer's disease.

In addition, the amyloid-beta plaque disaggregation ability mentioned above shows high efficacy even at low concentrations, and shows high efficiency with low toxicity.

### DESCRIPTION OF DRAWINGS

FIG. 1 is a graph showing (a) a library of sulfonic acid-based compounds as amyloid-beta (AB) plaque disaggregating agent candidates; and (b) results of comparing the amyloid-beta plaque disaggregation efficacy of each compound using Thioflavin T (mean±SEM, n=3).

FIG. 2 is a graph showing results of confirming the amyloid-beta plaque disaggregation efficacy of a compound (ANA) according to the present disclosure according to (a) a concentration change and (b) a pH change using Thioflavin T (mean35 SEM, n=3).

FIG. 3 shows results of confirming the disaggregation ability of amyloid-beta plaques according to the presence or absence of treatment of the compound (ANA) according to the present disclosure by (a) transmission electron microscopic and (b) confocal microscopic images (scale bar: 200 nm; excitation and emission channels: 520 nm, 548 to 617 nm).

FIG. 4 is a schematic diagram showing a manufacturing process of Biotin-CaCl₂-ANA-pSiNPs (BCAP) as a brain-targeting amyloid-beta plaque disaggregation nano platform (APDN) using the compound (ANA) according to the present disclosure; pSiNPs: porous silicon nanoparticles, CAP: pSiNPs loaded with ANA and sealed with calcium chloride (CaCl₂-ANA-pSiNPs), BCAP: CAP functionalized with biotin (Biotin-CaCl₂-ANA-pSiNPs).

FIG. 5 shows results of confirming changes in chemical properties according to a manufacturing step of a brain-targeting amyloid-beta plaque disaggregation nano platform using a compound (ANA) according to the present disclosure (ANA-pSiNP: pSiNP loaded only with ANA before CaCl₂ sealing, CAP: CaCl₂-ANA-pSiNPs, BCAP: Biotin-CaCl₂-ANA-pSiNPs) through (a) an average hydrodynamic diameter, (b) a zeta potential (polydispersity index: <0.3), (c) an ATR-FTIR spectroscopy spectrum (ν=stretching), and (d) a transmission electron microscope image (scale bar: 100 nm) (mean±SEM, n=4).

FIG. 6 shows (a) an emission intensity plot of ANA at 420 nm (excitation wavelength: 338 nm) showing results of confirming a concentration of the compound (ANA) dissolved from BCAP as a brain-targeting amyloid-beta plaque disaggregation nano platform using the compound

(ANA) according to the present disclosure and (b) a ThT emission spectrum according to an incubation time (excitation wavelength: 413 nm) showing the amyloid-beta plaque disaggregation ability using the same using Thioflavin T as an amyloid-beta plaque probe.

FIG. 7 shows results of confirming an in vivo toxicity response of a brain-targeting amyloid-beta plaque disaggregation nano platform using a compound (ANA) according to the present disclosure by hemolysis analysis (mean±SEM, n=3, ****<0.0001 (compared to Triton X-100 group)).

FIG. 8 shows (a) a FTIS image (channel: 390 to 490 nm excitation, 500 to 550 nm detection channel) showing results of confirming an in vivo distribution ratio of the brain-targeting amyloid-beta plaque disaggregation nano platform using the compound (ANA) according to the present disclosure by tracking the fluorescence of the compound (ANA) and (b) a graph showing a radiation efficiency plot of each organ in the FTIS image (mean±SEM, n=4, **<0.01 (compared to a PBS group), ##<0.01 (compared to an ANA group)).

FIG. 9 is a schematic diagram showing fabrication of an Alzheimer's disease (AD) mouse model and an animal experiment using the same to confirm the amyloid-beta plaque disaggregation efficacy of the brain-targeting amyloid-beta plaque disaggregation nano platform using the compound (ANA) according to the present disclosure.

FIG. 10 is a graph showing results of a Y-maze animal behavior (voluntary alternation) experiment to confirm the memory improvement efficacy of a compound (ANA) according to the present disclosure and a brain-targeting amyloid-beta plaque disaggregation nano platform using the same (mean±SEM, n=8, **<0.01 (compared to a sham group), #<0.05 (compared to a PBS group)).

FIG. 11 is a graph showing results of a passive avoidance test (PAT) animal behavior experiment to confirm the memory improvement efficacy of a compound (ANA) according to the present disclosure and a brain-targeting amyloid-beta plaque disaggregation nano platform using the same (mean±SEM, n=8, ****<0.0001 (compared to a sham group), ###<0.001 (compared to a PBS group)).

FIG. 12 is a graph showing results of ELISA analysis of interleukin 6 (IL-6), as a result of confirming responses to immune factors of a compound (ANA) according to the present disclosure and a brain-targeting amyloid-beta plaque disaggregation nano platform using the same (mean±SEM, n=4, *<0.05 (compared to a sham group), #<0.05 (compared to a PBS group)).

FIG. 13 is a fluorescence image of a hippocampus section stained with ThS showing results of confirming the amyloid-beta plaque disaggregation ability of a compound (ANA) according to the present disclosure and a brain-targeting amyloid-beta plaque disaggregation nano platform using the same in the hippocampus region of the actual brain (red arrow: location of amyloid-beta plaque).

FIG. 14 is an image showing mechanisms of a compound (ANA) according to the present disclosure and a brain-targeting amyloid-beta plaque disaggregation nano platform using the same.

### BEST MODE

Hereinafter, the present disclosure will be described in detail.

In an aspect, the present disclosure provides a compound represented by the following Chemical Formula 1:

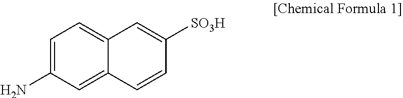 [SMILES: Nc1ccc2cc(S(=O)(=O)O)ccc2c1]

In the example of the present disclosure, the compound represented by Chemical Formula 1, 6-amino-2-naphthalenesulfonic acid (ANA), was discovered as a novel amyloid-beta plaque disaggregating agent, and the compound was named ANA.

The compound represented by Chemical Formula 1 of the present disclosure may also be used in the form of a hydrate thereof, a solvate thereof, or a pharmaceutically acceptable salt thereof.

In addition, ANA was loaded onto a porous silicon nanoparticle, which is one of nano carriers, and coated using calcium chloride (CaCl₂), and then surface-treated with biotin-polyethyleneglycerol (biotin-PEG) to develop a brain-targeting amyloid-beta plaque disaggregation nano platform, which was named BCAP.

Therefore, the present disclosure provides a nano platform including the compound represented by Chemical Formula 1, a hydrate thereof, a solvate thereof, or a pharmaceutically acceptable salt thereof.

The nano platform according to the present disclosure may be configured using other materials using the ANA.

Therefore, the nano platform of the present disclosure is characterized in that the compound represented by Chemical Formula 1, a hydrate thereof, a solvate thereof, or a pharmaceutically acceptable salt thereof is loaded onto the porous nanoparticles.

The porous nanoparticles may be used without limitation as long as the porous nanoparticles are nanoparticles known in the art that are movable into a living body. In one example of the present disclosure, porous silicon nanoparticles were used, but are not limited thereto.

In addition, the nano platform according to the present disclosure is characterized in that the surface is modified with a brain targeting moiety. The brain targeting moiety may be biotin-polyethylene glycol (biotin-PEG), but is not limited thereto. More preferably, the porous nanoparticles may be porous silicon nanoparticles of which the surface is sealed with calcium chloride and functionalized with biotin-polyethylene glycol (biotin-PEG).

Further, the present disclosure provides a manufacturing method of a nano platform according to the present disclosure including: preparing porous silicon nanoparticles; loading the compound represented by Chemical Formula 1, a hydrate thereof, a solvate thereof, or a pharmaceutically acceptable salt thereof onto the porous silicon nanoparticles; sealing the surface of the loaded porous silicon nanoparticles with calcium chloride; and surface-modifying the coated porous silicon nanoparticles with biotin-polyethyleneglycerol (biotin-PEG).

In the manufacturing method of the present disclosure, the sealing with calcium chloride is performed by reacting the porous silicon nanoparticles with calcium or magnesium.

The amyloid-beta plaque disaggregation efficacy of the present disclosure is to specifically disaggregate amyloid-beta plaques, and may appear anywhere in the body when the material is administered into the body. Preferably, the amyloid-beta plaque disaggregation efficacy exhibits targeting activity to the brain, and the corresponding characteristic may mean a characteristic in which significantly higher efficacy is exhibited in the brain than in the rest of the body.

The brain targeting activity exhibited by the compound and the nano platform according to the present disclosure may mean delivering a material specifically to the brain.

The compound and the nano platform according to the present disclosure may bind to amyloid-beta plaques and may be involved in the amyloid-beta plaques. Preferably, the compound and the nano platform may disaggregate the amyloid-beta plaques.

In particular, the nano platform of the present disclosure showed a characteristic that may specifically target the brain, and therefore, it was confirmed that the nano platform may exhibit an effect of improving memory loss, an increase in immune factors, etc. induced by the amyloid-beta plaques more efficiently than ANA (see Examples 5 to 9). In addition, the compound and the nano platform according to the present disclosure may improve high immune levels induced by the amyloid-beta plaques.

Further, the present disclosure provides a composition for disaggregating amyloid-beta plaques, including the compound represented by Chemical Formula 1, a hydrate thereof, a solvate thereof, or a pharmaceutically acceptable salt thereof.

Further, the present disclosure provides a composition for disaggregating amyloid-beta plaques, including a nano platform according to the present disclosure.

The compound and the nano platform according to the present disclosure may disaggregate the amyloid-beta plaques in a concentration-dependent manner.

The compound and the nano platform according to the present disclosure may disaggregate the amyloid-beta plaques at pH 3, 5, 7, and 9, and more preferably, may exhibit the highest efficacy at pH 7.4.

The nano platform according to the present disclosure may exhibit almost no toxicity in vivo.

The composition for disaggregating the amyloid-beta plaques may further include at least one selected from a solvent, an acid, a base, and a buffer solution. The composition for disaggregating the amyloid-beta plaques may be prepared by adding the above-described compound to a solvent, a buffer solution, or a mixture thereof, and adding an acid or a base thereto. In addition, the composition for disaggregating the amyloid-beta plaques may additionally include other additives that may be used in the art. The contents of the solvent, the acid, the base, and the buffer solution included in the composition may be appropriately adjusted depending on the required performance. The solvent may include water, THF, methanol, ethanol, an aqueous HI solution, N,N-dimethylformamide, or a combination thereof. Alternatively, the composition may include a combination with other drugs and chemical molecules. The drug may include drugs having the amyloid-beta plaque disaggregation efficacy, but is not limited thereto, and include any drug or chemical sample that may be used in the art.

Further, the present disclosure provides a pharmaceutical composition for preventing or treating a disease caused by amyloid-beta plaques, including the compound represented by Chemical Formula 1, a hydrate thereof, a solvate thereof, or a pharmaceutically acceptable salt thereof.

Further, the present disclosure provides a pharmaceutical composition for preventing or treating a disease caused by amyloid-beta plaques, including a nano platform according to the present disclosure.

In the pharmaceutical composition of the present disclosure, the disease is preferably a disease caused by the accumulation of amyloid-beta plaques. More preferably, the disease caused by the amyloid-beta plaques may be at least one selected from the group consisting of Alzheimer's disease, Parkinson's disease, stroke, Down syndrome, amyloid angiopathy, systemic amyloidosis, Dutch amyloidosis, inclusion body myositis, Creutzfeldt-Jakob disease, Kennedy's disease, Amyotrophic Lateral Sclerosis, Fronto-Temporal Dementia, Cortico-Basal Degeneration, Huntington's disease, senile dementia of the Alzheimer type, Lewis body dementia, vascular dementia, mild cognitive impairment, and age-related memory impairment, but is not limited thereto.

Accordingly, the pharmaceutical composition of the present disclosure may be administered to a “subject” that has developed or is likely to develop the disease caused by the amyloid-beta plaques, and the “subject” may mean all animals including humans.

As used in the present disclosure, the term “prevention” refers to any action that suppresses the symptoms of a specific disease or delays its progression by administering the composition of the present disclosure.

As used in the present disclosure, the term “treatment” refers to any action that improves or beneficially changes the symptoms of a specific disease by administering the composition of the present disclosure.

The pharmaceutical composition of the present disclosure may further include an adjuvant in addition to the active ingredient. The adjuvant may be used with any adjuvant known in the art without limitation, but further include, for example, a Freund's complete adjuvant or an incomplete adjuvant to increase the effect thereof.

The pharmaceutical composition according to the present disclosure may be prepared in the form of incorporating the active ingredient into a pharmaceutically acceptable carrier. Here, the pharmaceutically acceptable carrier includes carriers, excipients and diluents commonly used in a pharmaceutical field. The pharmaceutically acceptable carrier that may be used in the pharmaceutical composition of the present disclosure is not limited thereto, but may include lactose, dextrose, sucrose, sorbitol, mannitol, xylitol, erythritol, maltitol, starch, acacia gum, alginate, gelatin, calcium phosphate, calcium silicate, cellulose, methyl cellulose, polyvinylpyrrolidone, water, methyl hydroxybenzoate, propyl hydroxybenzoate, talc, magnesium stearate, and mineral oil.

The pharmaceutical composition according to the present disclosure may be formulated and used in the form of oral formulations such as powders, granules, tablets, capsules, suspensions, emulsions, syrups, aerosols, etc., external preparations, suppositories, and sterile injectable solutions according to a conventional method.

The formulations may be prepared by using diluents or excipients, such as a filler, an extender, a binder, a wetting agent, a disintegrating agent, a surfactant, etc., which are generally used. Solid formulations for oral administration include tablets, pills, powders, granules, capsules, etc., and these solid formulations may be prepared by mixing at least one or more excipients, for example, starch, calcium carbonate, sucrose, lactose, gelatin, etc. with the active ingredient. Further, lubricants such as magnesium stearate and talc may be used in addition to simple excipients. Liquid formulations for oral administration may correspond to suspensions, oral liquids, emulsions, syrups, etc., and may include various excipients, for example, a wetting agent, a sweetener, an aromatic agent, a preserving agent, etc., in addition to the commonly used diluents, such as water and liquid paraffin. Formulations for parenteral administration include sterile aqueous solutions, non-aqueous solvents, suspensions, emulsions, lyophilized agents, and suppositories. As the non-aqueous solvent and the suspension, propylene glycol, polyethylene glycol, vegetable oil such as olive oil, injectable ester such as ethyl oleate, etc. may be used. As the base material of the suppository, witepsol, Tween 61, cacao butter, laurinum, glycerogelatin, etc. may be used.

The pharmaceutical composition of the present disclosure may be administered to a subject through various routes. All methods of administration may be expected, and the pharmaceutical composition may be administered by, for example, oral, intravenous, intramuscular, subcutaneous, and intraperitoneal injection.

The dose of the pharmaceutical composition according to the present disclosure is selected in consideration of the age, body weight, sex, and physical condition of the subject. It is obvious that the concentration of the active ingredient included in the pharmaceutical composition may be selected in various ways depending on a subject.

In addition, according to one example of the present disclosure, it was confirmed that the nano platform developed using the compound of the present disclosure may improve a decreased memory of mice induced by amyloid-beta plaques.

Accordingly, the present disclosure provides a pharmaceutical composition for preventing or improving memory loss, including the compound represented by Chemical Formula 1, a hydrate thereof, a solvate thereof, or a pharmaceutically acceptable salt thereof.

Further, the present disclosure provides a pharmaceutical composition for preventing or improving memory loss, including a nano platform according to the present disclosure.

In the present disclosure, the memory loss is induced by amyloid-beta plaques, and preferably induced by the accumulation of amyloid-beta plaques.

Further, the present disclosure provides a method for disaggregating amyloid-beta plaques, including administering to a subject the compound represented by Chemical Formula 1, a hydrate thereof, a solvate thereof, or a pharmaceutically acceptable salt thereof.

Further, the present disclosure provides a method for disaggregating amyloid-beta plaques, including administering to a subject a nano platform according to the present disclosure.

In the method for disaggregating the amyloid-beta plaques according to the present disclosure, the method includes both a method performed in vitro and a method performed in a cell or in vivo, and is not limited in a specific part, and includes any method that may be used as a biological sample in the art.

Further, the present disclosure provides a method for preventing or treating a disease caused by amyloid-beta plaques, including administering to a subject the compound represented by Chemical Formula 1, a hydrate thereof, a solvate thereof, or a pharmaceutically acceptable salt thereof.

Further, the present disclosure provides a method for preventing or treating a disease caused by amyloid-beta plaques, including administering to a subject a nano platform according to the present disclosure.

In the present disclosure, preferably, the disease is a disease caused by the accumulation of amyloid-beta plaques. More preferably, the disease caused by the amyloid-beta plaques may be at least one selected from the group consisting of Alzheimer's disease, Parkinson's disease, stroke, Down syndrome, amyloid angiopathy, systemic amyloidosis, Dutch amyloidosis, inclusion body myositis, Creutzfeldt-Jakob disease, Kennedy's disease, Amyotrophic Lateral Sclerosis, Fronto-Temporal Dementia, Cortico-Basal Degeneration, Huntington's disease, senile dementia of the Alzheimer type, Lewis body dementia, vascular dementia, mild cognitive impairment, and age-related memory impairment, but is not limited thereto.

Further, the present disclosure provides a method for preventing or improving memory loss, including administering to a subject the compound represented by Chemical Formula 1, a hydrate thereof, a solvate thereof, or a pharmaceutically acceptable salt thereof.

Further, the present disclosure provides a method for preventing or improving memory loss, including administering to a subject a nano platform according to the present disclosure.

Hereinafter, the present disclosure will be described in more detail by the following Examples. However, these Examples are only provided for illustrating the present disclosure, and the scope of the present disclosure is not limited by these Examples.

### Preparation Example 1. Preparation of

To prepare amyloid-beta plaques, amyloid-beta (Aβ) 1-42 (Anaspec, AS-20276, MW=4514.1, 1 mg) was dissolved in a 1× PBS buffer (pH 7.4, 1 mL) to a final concentration of 222 μM. The solution was incubated (200 rpm) at 37° C. for 3 days, and the formed amyloid-beta plaques (Aβ plaques) were used in the following in vitro experiment without additional purification.

### Preparation Example 2. Preparation of Animal Model

6-week-old male balb/c mice were purchased from DBEL (Incheon, Korea). Five mice (27×22×14 cm) were contained in each cage in an animal room with a constant temperature (23±1° C.) and relative humidity (60±10%), and a 12-hour light/dark cycle (lighting on from 07:30 to 19:30), and freely accessed to food and water. The mice were kept in the animal room for one week before the experiment. Animal treatment and maintenance were performed in accordance with the Animal Care and Use Guidelines of Kyung Hee University. All experimental protocols were approved by the Animal Laboratory Management Committee of Kyung Hee University (Approval No.: KHSASP-22-024).

### Reference Example 1. Statistical procssing

The data of Examples of the present disclosure were expressed as the mean +the standard error of the mean (S.E.M). ThS intensity was analyzed by an unpaired t-test. The IVIS data, hemolysis test, Ymaze test, PAT, and ELISA analysis results were analyzed by a Tukey's multiple comparison test and one-way analysis of variance (ANOVA). All statistical results were analyzed using Prism 8.0 software (GraphPad, La Jolla, CA, USA).

### Example 1. Confirmation of Effects of Hit Compounds Through Screening of Amyloid-Beta Plaque Disaggregating Agent Candidates

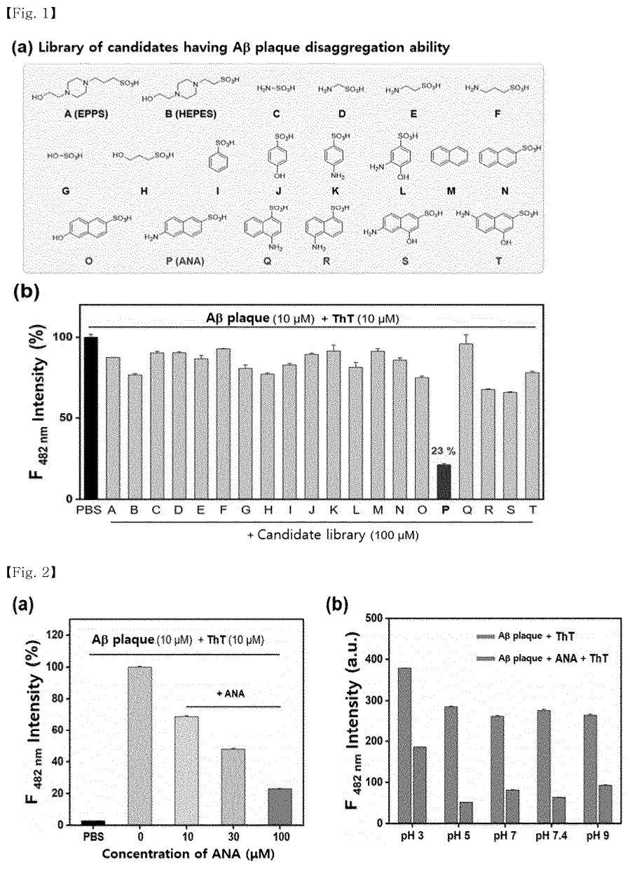

In order to discover compounds capable of effectively inducing the disaggregation of amyloid-beta plaques, the present inventors formed a library using various sulfonic acid compounds as candidates and then screened the candidates. The library included 20 compounds from A to T in Table 1 below, and a structure of each of amyloid-beta plaque disaggregating agent candidates included in the library was shown in (a) of FIG. 1.

TABLE 1

| Classification | Compound | Compound name |
|---|---|---|
| Aliphatic | A | 4-(2-hydroxyethyl)-1- |
| sulfonic acid | | piperazine |
| analogue | | propanesulfonic acid |
| | | (EPPS) |
| | B | 2-[4-(2- |
| | | hydroxyethyl)piperazin- |
| | | 1-yl]ethanesulfonic |
| | | acid (HEPES) |
| | C | Sulfamic acid |
| | D | Aminomethanesulfonic acid |
| | E | 2-Aminoethanesulfonic acid |
| | F | 2-Aminoethanesulfonic acid |
| | G | Sulfonic acid |
| | H | 3-Hydroxypropanesulfonic acid |
| Benzene | I | Benzenesulfonic acid |
| sulfonic acid | J | 4-Hydroxybenzenesulfonic acid |
| analogue | | |
| Benzene | K | Sulfanilic acid |
| sulfonic acid | | |
| analogue | | |
| | L | 3-amino-4- |
| | | hydroxybenzenesulfonic acid |
| Naphthalene | M | Naphthalene |
| sulfonic acid | | |
| analogue | | |
| | N | 2-Naphthalenesulfonic acid |
| | O | 2-Naphthol-6-sulfonic acid |
| | P | 6-Amino-2-naphthalenesulfonic |
| | | acid (ANA) |
| | Q | 4-Amino-1-naphthalenesulfonic |
| | | acid |
| | R | 5-Amino-1-naphthalenesulfonic |
| | | acid |
| | S | 6-Amino-4-hydroxy-2- |
| | | naphthalenesulfonic acid |
| | T | 6-Amino-1-naphthol-3-sulfonic |
| | | acid |

From the formed library, hit compounds were identified through fluorescence analysis using thioflavin T (ThT), a probe of amyloid-beta plaques. Specifically, each amyloid-beta plaque disaggregation candidate (100 μM) was mixed with amyloid-beta plaques (10 μM) and ThT (10 μM), and incubated at 37° C. for 24 hours, and then a change in fluorescence intensity of ThT at 482 nm was confirmed (excitation wavelength: 413 nm). The absorption and emission spectra of ThT were obtained using UV/vis absorption spectroscopy (Agilent, California, USA) and a spectrofluorophotometer (Shimadzu Corp., Kyoto, Japan).

The results of fluorescence analysis using thioflavin T to determine the amyloid-beta plaque disaggregation degree of amyloid-beta plaque disaggregating agent candidates were shown in a graph of (b) of FIG. 1. 6-amino-2-naphthalenesulfonic acid (ANA), corresponding to Compound P, showed the greatest fluorescence intensity change of 23% compared to a control group. Therefore, among A-T candidates, ANA, corresponding to Compound P, showed the highest amyloid-beta plaque disaggregation efficacy, which was a hit compound.

### Example 2. Confirmation of Amyloid-Beta Plaque Disaggregation Efficacy of Compound (ANA) Under Various Conditions

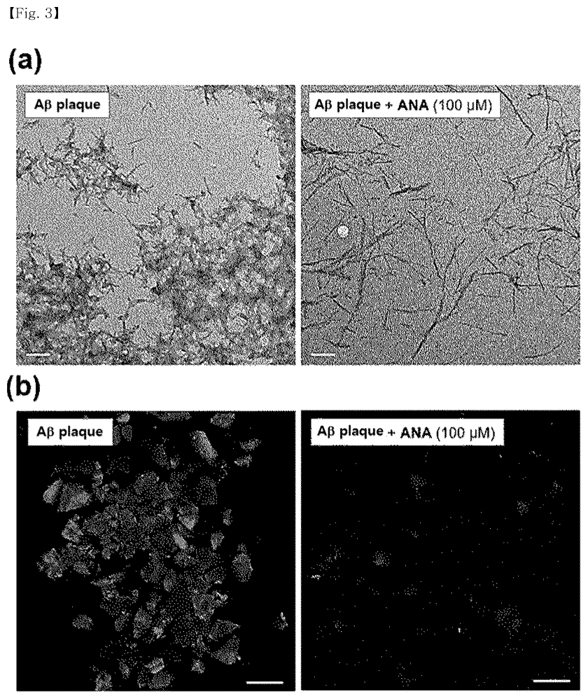

For Compound P (ANA), a hit compound confirmed in Example 1, the amyloid-beta plaque disaggregation efficacy under various concentration conditions and pH conditions was confirmed through ThT assay in the same manner as in Example 1, and an emission intensity plot of ThT (10 μM) at 482 nm was shown in FIG. 2 (excitation wavelength: 413 nm).

(a) of FIG. 2 is a graph showing results of changes in fluorescence intensity of ThT after the reaction of ANA at various concentrations (10 to 100 μM) and amyloid-beta plaques (10 μM). The ANA showed amyloid-beta plaque disaggregation efficacy in a concentration-dependent manner at a total concentration of 10 μM to 100 μM, and showed significantly high amyloid-beta plaque disaggregation efficacy even at a low concentration of 10 μM.

(b) of FIG. 2 is a graph showing fluorescence changes using ThT to confirm the amyloid-beta plaque disaggregation efficacy of ANA under various pH conditions. The ANA showed amyloid-beta plaque disaggregation efficacy at all pH conditions of pH 3, 5, 7, 7.4, and 9, and in particular, showed the highest amyloid-beta plaque disaggregation efficacy at pH 7.4, which was a pH condition of the brain.

### Example 3. Visual Confirmation of Amyloid-Beta Plaque Disaggregation Efficacy of Compound (ANA)

After confirming in vitro that ANA may disaggregate amyloid-beta plaques, visual images were confirmed using transmission electron microscopy and confocal microscopy to directly confirm the disaggregation visually. The results were shown in FIG. 3.

For TEM analysis, amyloid-beta plaques (20 μM) or amyloid-beta plaques (20 μM) and a compound (ANA, 100 μM) were treated on copper grids at room temperature. Each grid treated with samples (Aβ plaques or Aβ plaques+ANA) was washed three times with DI H₂O and then stained with uranyl acetate (1% w/v DI H₂O, 5 μL) for 1 minute. The uranyl acetate was removed, the grids were dried at room temperature for 20 minutes, and then the morphology of amyloid-beta plaques was analyzed by transmission electron microscopic images (TEM, Tecnai, G2 F30ST, FEI Company, OR, USA).

(a) of FIG. 3 shows TEM images of amyloid-beta plaques before and after ANA treatment, in which it was confirmed that the amyloid-beta plaques that had formed the plaques were disaggregated and released into monomers after ANA (100 M) treatment.

In addition, to evaluate the presence of amyloid-beta plaques (AB plaques) and a change in the disaggregation morphology of amyloid-beta plaques according to incubation with ANA for 24 hours, thioflavin S (ThS), a probe of amyloid-beta plaques, was additionally treated, and the change thereof was confirmed by fluorescence analysis using a confocal microscope. The samples (Aβ plaques, Aβ plaques +ANA) were added to 1 mL of a ThS (0.5%) solution, and each mixture was pipetted onto a slide glass and dried overnight at room temperature. Then, fluorescence images were obtained using a laser scanning confocal microscope (CLSM, LSM800, Zeiss, Oberkochen, Germany). Confocal images were obtained under excitation at 520 nm (laser power: 3.00%) using a detector (GaAsP, detector gain: 680 V, detector wavelength: 548 to 617 nm).

(b) of FIG. 3 shows confocal microscopic images of amyloid-beta plaques before and after ANA treatment, in which Thioflavin S (ThS), a probe of amyloid-beta plaques, was additionally treated to track the fluorescence, and the changes were confirmed. The fluorescence intensity of ThS, which was increased when only amyloid-beta plaques were present, was significantly reduced after ANA treatment, and these results visually confirmed that ANA could disaggregate amyloid-beta plaques.

### Example 4. Manufacturing Process of BCAP as Brain-Targeting Amyloid-Beta Plaque Disaggregation Nano Platform (APDN)

Amyloid-beta plaques, which are expected to be pathogenic materials of Alzheimer's disease, are evenly distributed throughout our body, but only amyloid-beta plaques that substantially exist in the brain may cause toxicity to the brain and induce Alzheimer's disease. Therefore, an amyloid-beta plaque disaggregating agent for treating Alzheimer's disease needs to disaggregate amyloid-beta plaques in the brain.

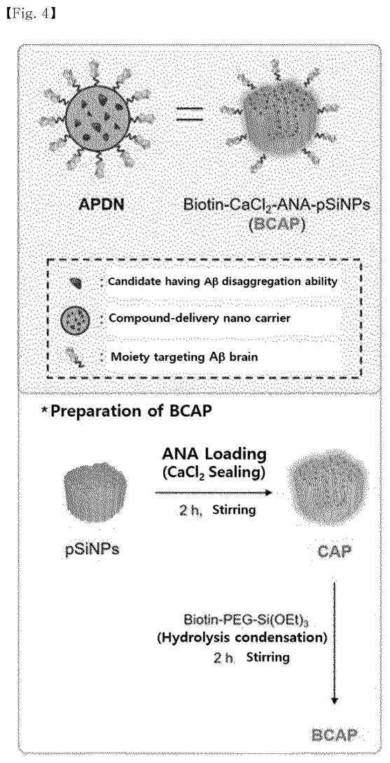

To this end, the present inventors developed an amyloid-beta plaque disaggregation nano platform (APDN) that may deliver the compound (ANA) to the brain (see FIG. 4). The APDN was designed to consist of an amyloid-beta plaque disaggregating agent, a nano-carrier capable of delivering the disaggregating agent, and a brain-targeting molecule.

As shown in FIG. 4, the present inventors developed a brain-targeting amyloid-beta plaque disaggregation nano platform by using ANA as an amyloid-beta plaque disaggregating agent, loading the ANA onto porous silicon nanoparticles (pSiNPs), which were nano-carriers, in the presence of calcium chloride (CaCl₂), then coating the porous silicon nanoparticles the with calcium chloride (CaCl₂), and surface-modified the porous silicon nanoparticles with biotin-polyethyleneglycerol (biotin-PEG), which was named Biotin-CaCl₂-ANA-pSiNPs (BCAP). A specific manufacturing method of BCAP was as follows.

### 4-1. Preparation of Porous Silicon Nanoparticles (pSiNPs)

Porous silicon nanoparticles (pSiNPs) applied as nano carriers were prepared by electrochemical etching of p⁺⁺-type single-crystal silicon wafers (highly doped with boron).

Specifically, (i) a silicon wafer was electrochemically etched in an electrolyte (48% aqueous HF/absolute EtOH, 3:1, v: v). (ii) The silicon wafer was electrochemically etched in a porous sacrificial layer with a 3:1 (v: v) 48% aqueous HF/ethanol electrolyte to prepare a porous silicon (pSi) layer. (iii) The generated pSi layer was removed using a 2 M potassium hydroxide solution (KOH). At this time, a perforation etching waveform was applied by repeating a current density pulse of 46 mA cm⁻² or less for 1.8 seconds and a current density pulse of 334 mA cm⁻² or more for 0.4 second for 300 cycles. Then, a porous Si film was separated from the silicon wafer by applying a current density pulse of 3 mA cm⁻² for 300 seconds in a 7.5% HF aqueous solution in ethanol. (iv) The freestanding pSi film was placed in a sealed glass vial (22.18 mL size, VWR, product number 66011-143, Radnor, PA, USA) containing deionized water (DI H₂O, 6 mL) and disrupted into nanoparticles in an ultrasonic bath (VWR, Model No. VWRA142-0307, Radnor, PA, USA) for 24 hours. (v) The nanoparticles obtained by sonication were further incubated in DI H₂O (12 mL) at 25°° C. for 3 days to form a silicon oxide layer on the surface, and then the porous silicon nanoparticles (pSiNPs) were filtered through a 0.22 μm syringe filter (Millipore, Millex syringe filter device, 220 nm, model number SLGP033RS, Burlington, MA, USA). (vi) The filtered pSiNPs were collected by centrifugation (14,000 rpm, 15 min) and re-dispersed/washed (3 times) with EtOH.

### 4-2. Preparation of BCAP

The porous silicon nanoparticles (pSiNPs) prepared in Example 4-1 were dispersed in DI H₂O to prepare a solution. Next, an ANA stock solution (10 mg/mL DMSO, 100 μL) was added to a pSiNP (1 mg) solution dispersed in DI H₂O (400 μL). The mixture reacted with a 4 M calcium chloride (CaCl₂) stock solution (500 μL) using a vortex mixer (600 rpm, Scientific industries, Inc., VortexGenie 2, Model No. SI-0246, Bohemia, NY, USA) at room temperature for 2 hours to generate porous silicon nanoparticles (CaC12-ANA-pSiNPs; CAP) loaded with ANA into the pores and surface-coated with calcium chloride. The CaCl₂-ANA-pSiNPs (CAP) were collected by centrifugation (14,000 rpm, 15 min) and washed three times with DI H₂O (1 mL). Thereafter, the CAP solution dispersed in EtOH (900 μL) was added with a silane-PEG-Biotin (5000 Da) stock solution (100 μL, 10 mg/mL, EtOH) and mixed using a vortex mixer at room temperature for 2 hours to functionalize the surface of the CAP with biotin-polyethylene glycol (PEG). After 2 hours, the generated Biotin-CaCl₂-ANA-pSiNPs (CAP surface-modified with biotin-PEG; BCAP) were washed three times using EtOH (1 mL) and collected by centrifugation (14,000 rpm, 15 min).

### Example 5. Confirmation of Chemical Properties of BCAP

BCAP, a brain-targeting amyloid-beta plaque disaggregation nano platform, was prepared from porous silicon nanoparticles (pSiNPs) through stepwise changes. In order to confirm the chemical properties and structural changes of BACP according to these stepwise changes, the average hydrodynamic diameter (nano particle size) and zeta potential of each of pSiNPs, CAP, and BCAP were analyzed by dynamic light scattering (DLS) using a Malvern Instruments Zetasizer Nano, and an infrared absorption spectrum was analyzed using attenuated total reflection Fourier transform infrared (ATR-FTIR) spectroscopy, respectively. Image-based analysis using transmission electron microscopy (TEM) was performed to observe the structural changes of porous silicon nanoparticles (ANA_pSINPs) loaded only with ANA before coating with each agent of pSiNPs, CAP, and BCAP and CaCl₂.

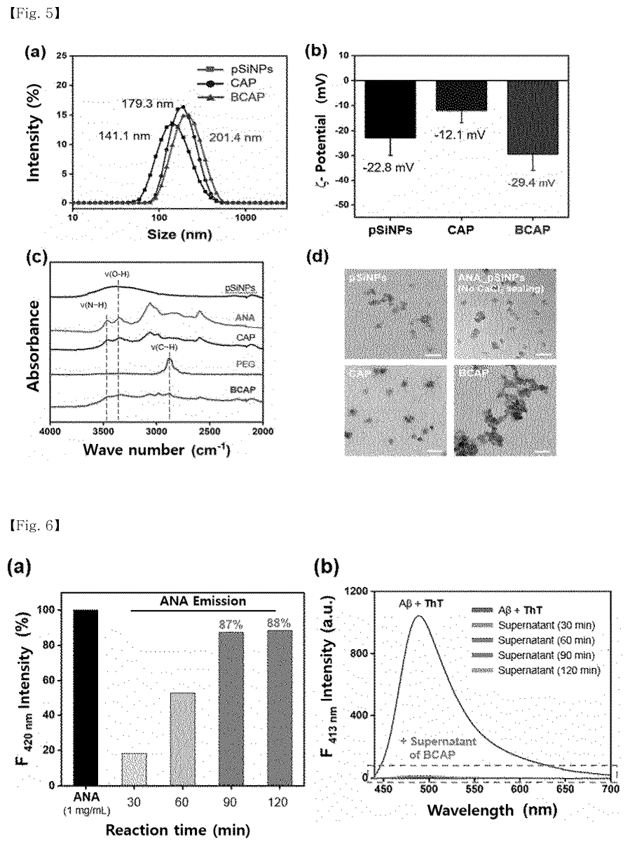

(a) of FIG. 5 shows changes in particle size of each of pSiNPs, CAP, and BCAP through stepwise changes from porous silicon nanoparticles (pSiNPs) to BCAP, in which it was confirmed that the average hydrodynamic diameter size gradually increased with each step.

(b) of FIG. 5 shows results of confirming the zeta potential according to a stepwise change of each of pSiNPs, CAP, and BCAP analyzed by dynamic light scattering (DLS), in which the potential, which was initially −22.8 mV, was changed to −12.1 mV by calcium chloride after coating with CaCl₂, and then changed to −29.4 mV after surface modification with biotin-PEG, so that the negative charge was strengthened.

(c) of FIG. 5 shows results of confirming changes in functional groups according to a stepwise reaction using ATR-Fourier transform infrared spectroscopy (ATR-FTIR spectroscopy). In the results, it was confirmed that BCAP showed an O—H group observed in porous silicon nanoparticles, an N—H group observed in ANA, and a C—H group observed in biotin-PEG, and thus these functional groups were added step-by-step.

(d) of FIG. 5 shows transmission electron microscopy results according to changes at each step, in which it was confirmed that opened pores of the porous silicon nanoparticles were observed until the step where ANA was loaded, but the pores were closed after coating with calcium silicate.

With these results, it was confirmed that the brain-targeting amyloid-beta plaque disaggregation nano platform using porous silicon nanoparticles was formed as an expected schematic diagram.

### Example 6. Confirmation of ANA Release from BCAP and Verification of Amyloid-Beta Plaque Disaggregation Efficacy of Released ANA

The previously formed BCAP indirectly showed that ANA was loaded, but it was not confirmed how much ANA was loaded and whether the efficacy of ANA was denatured during the loading process. For this verification, the formed BCAP was released in PBS and then incubated at 37° C. in a shaking incubator to determine the amount of ANA released over time.

Specifically, BCAP (1 mg) was dispersed in PBS (1 mL, pH 7.4) and the solution was cultured at 37° C. for 120 minutes (time interval: 30 minutes each, 0 to 120 minutes). At each time interval (30 minutes, 60 minutes, 90 minutes, and 120 minutes), BCAP was removed from the water phase by centrifugation at 14,000 rpm for 15 minutes. To measure a release profile of ANA loaded into BCAP, the supernatant obtained by the centrifugation was placed in a 1 cm standard quartz cell (internal volume 1 mL, Hellma Analytics, Germany), and the fluorescence signal of ANA was tracked using a spectrofluorometer (Shimadzu Corp. RF-6000, Kyoto, Japan) to calculate the amount of ANA released from BCAP at 420 nm, and the emission intensity plot (excitation wavelength: 338 nm) was measured.

(a) of FIG. 6 shows results of confirming the release amount of ANA over time. Approximately 0.87 mg/mL of ANA was released from BCAP only within 90 minutes, and the maximum loading amount was confirmed to be approximately 0.88 mg/mL.

(b) of FIG. 6 shows an emission spectrum of ThT (10 μM) obtained at each incubation time in the presence of the supernatant (containing ANA) of BCAP (PBS buffer, pH 7.4, 25° C.) (excitation wavelength: 413 nm). In the corresponding results, it was confirmed that ANA still has the amyloid-beta plaque disaggregation efficacy, and the results were also concentration-dependent.

### Example 7. Confirmation of Biotoxicity of BCAP Through Hemolytic Reaction

Before conducting an experiment on actual animals with BCAP, it should be confirmed that the animals did not exhibit toxicity. Therefore, for this verification, the present inventors collected blood from the animals of Preparation Example 2, separated only red blood cells, treated BCAP at different concentrations, reacted, and then performed a hemolytic test. Specifically, the blood was collected from the heart of a mouse anesthetized with isofluorane. Red blood cells (RBCs) were extracted by centrifugation at 4° C. (3,000 rpm, 3 min) and washed with 1× cold PBS (twice). ANA or BCAP at concentrations of 0.03, 0.1, 0.3, 1, and 3 mg/mL were treated at a 8% working concentration (v/v) in purified RBCs (cold 1× PBS).

0.1% (working concentration, v/v) Triton X-100 was used as a positive control group. The mixture was incubated in a shaking incubator (200 rpm, 37° C.) for 1 hour and then centrifuged at 3,000 rpm at 4° C. The hemolysis activity of the supernatant was measured under absorption at 450 nm.

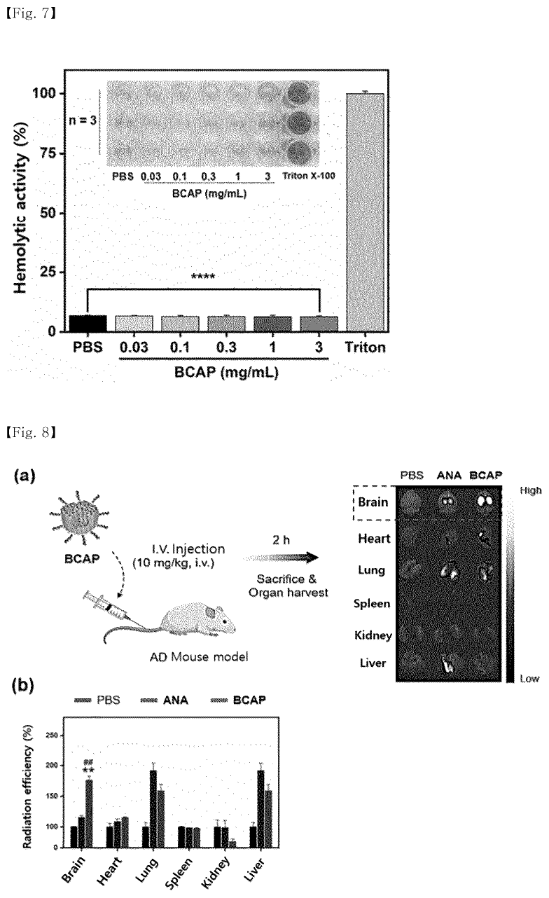

As a result, as shown in FIG. 7, BCAP did not show any toxicity reaction at all concentrations of 0.03 to 3 mg/mL. Based on the results of the experiment, the animal experiment was conducted at a concentration of 0.25 mg/mL.

### Example 8. Confirmation of Brain Targeting Efficacy of BCAP

Before confirming the efficacy of BCAP for treating Alzheimer's disease, it is necessary to prove the brain targeting efficacy of BCAP. Therefore, the present inventors administered ANA and BCAP (10 mg/kg, i.v.) to Alzheimer's disease-induced mice (Example 9-1), and then anesthetized the mice after 2 hours of drug in vivo circulation time, transcardially perfused with PBS (pH 7.4), and fixed with 4% paraformaldehyde. Major organs (brain, heart, lung, spleen, kidney, and liver) were extracted from the mice and the fluorescence intensities of each organ were compared and analyzed. A fluorescence tissue imaging system (FTIS, VISQUE InVivo Elite, Vieworks Co. Ltd., Korea) was used for in vitro tissue fluorescence imaging, and the imaging experiment was performed in a darkroom. The fluorescence intensity data were collected by tracking the fluorescence signal of ANA (channel: 390 to 490 nm excitation, 500 to 550 nm detection channel).

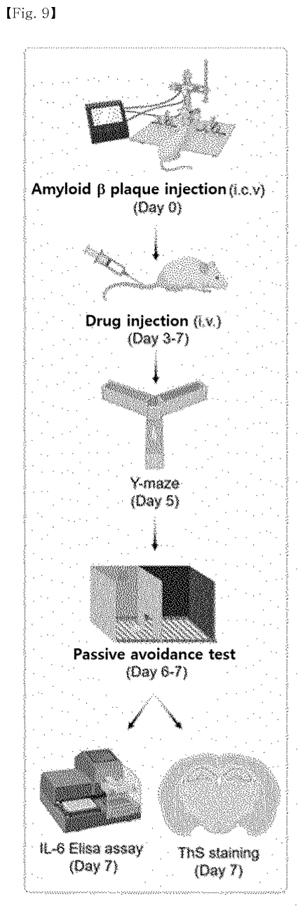

(a) of FIG. 8 is a schematic diagram of the experiment and an image result showing the fluorescence intensities in the extracted organs, and (b) of FIG. 8 is a graph of the fluorescence intensities confirmed as a result of fluorescence image. The results of the experiment were confirmed by tracking the fluorescence of ANA.

In the results of the experiment, it was confirmed that BCAP showed a significant difference only in the brain compared to when only ANA was administered, and could target the brain.

### Example 9. Confirmation of Efficacy of BCAP in Treating Alzheimer's Disease in Animals

The present inventors attempted to confirm whether BCAP showed an Alzheimer's disease treatment effect through the amyloid-beta plaque disaggregation efficacy. In order to confirm the efficacy of BCAP in treating Alzheimer's disease in mice, an Alzheimer's disease mouse model induced by amyloid-beta plaques was fabricated, and animal behavioral experiments such as Y-maze (day 5) and passive avoidance (days 6 and 7) experiments were performed while administering BCAP (days 3 to 7, daily administration). Thereafter, the mice were sacrificed to analyze interleukin 6 (IL-6) levels (day 7) and stain brain tissue using ThS (day 7). A schematic diagram of the analysis experiment was shown in FIG. 9.

### 9-1. Alzheimer's Disease Mouse Model

To fabricate an Alzheimer's disease mouse model induced by amyloid-beta plaques, the amyloid-beta plaques (222 μM) of Preparation Example 1 were stereotaxically administered using a Hamilton microsyringe and intracerebroventricularly (−0.2 mm anteroposterior (AP), −1 mm mediolateral (ML), and −2.4 mm dorsoventricular (DV), intracerebroventricular; i.c.v.) injected to mice anesthetized with isoflurane (Preparation Example 2) (1 μL/min, 5 min). The stereotaxic administration was performed in a separate heating room where a heating system controlled the body temperature of the mouse (maintained at 36 to 37° C.). Since a decrease in body temperature during anesthesia may induce tau phosphorylation, the temperature was regularly monitored using a thermometer. The injection of the drug (PBS, ANA, CAP or BCAP) (10 mg/kg, i.v.) was performed daily from 3 to 7 days after injection of amyloid-beta plaques.

### 9-2. Y-Maze Test

To determine whether the brain-targeting amyloid-beta plaque disaggregation nano platform of the present disclosure may improve memory loss induced by amyloid-beta plaques in an Alzheimer's disease mouse model, a Y-maze test was performed.

The Y-maze consisted of three dark opaque polyvinyl plastic arms (40×3×12 cm) arranged at a 120-degree angle from each other (A, B, and C). The last administration of PBS, ANA, CAP, and BCAP was performed 1 hour before the Y-maze test, whereas a Sham group was treated with a vehicle solution instead of ANA. Each mouse was initially disposed at a random arm, and the arm entry order (e.g., ABC, CAB, etc.) was recorded over 8 minutes using a video camera-based Etho Vision System (Noldus, Netherlands). Between each test, residual odor and residues from the Y-maze arms were removed using a 70% EtOH spray. An actual alternation was defined as consecutive entries into all three arms A, B, and C (i.e., in the order of ABC, BAC, or CAB, but not BCC or CCA). An actual alternation was manually observed by a person who did not know an administration group type. The spontaneous alternation score (%) of each mouse was calculated as a ratio of the actual number of alternations to the possible number of alternations (defined by subtracting 2 from the total number of maze arm entries of mice) multiplied by 100, as shown in the following Equation.

$$Spontaneous alternation ( \% ) = \text{} [ ( actual number of alternations ) / ( total number of arm entries - 2 ) ] \times 100.$$

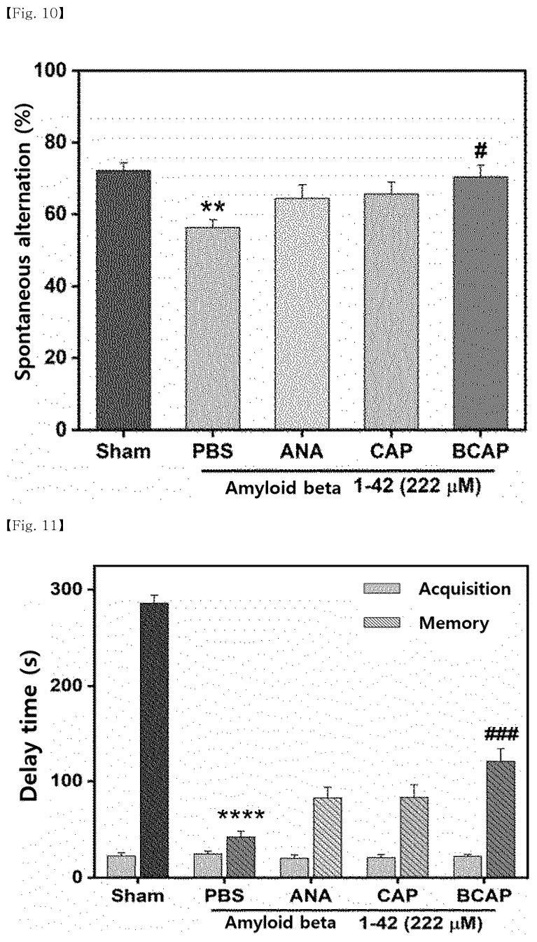

As shown in FIG. 10, as the Y-maze test result, it was confirmed that BCAP could significantly improve memory loss induced by amyloid-beta plaques.

### 9-3. Passive Avoidance Test

To measure long-term memory in an Alzheimer's disease mouse model, a passive avoidance test (PAT) was conducted.

The passive avoidance test was performed for two consecutive days for acquisition and retention trials in an apparatus consisting of two attached chambers (20×20×20 cm) connected by a door (5×5 cm). One chamber was a bright chamber (50 W light bulb) and the other chamber was a dark chamber without a light source, and 2 mm stainless steel bars were installed at 1 cm intervals on the bottom of each chamber.

On the first day, the acquisition trial was conducted. The mouse was gently placed in a light chamber, and the door between the light chamber and the dark chamber was opened after 10 seconds and automatically closed after the mouse entered the dark chamber. After the mouse entered the dark chamber, an electric shock (0.5 mA, 3 s) was applied to the mouse's foot through a stainless steel bar. If the mouse did not enter the dark chamber within 60 seconds, the mouse was forced to enter the dark chamber, and the latency was recorded as 60 seconds.

The retention trial was conducted 24 hours after the acquisition trial. The mouse was placed in the light chamber and the door was opened after 10 seconds under the same conditions as the acquisition trial. The time when it took for the mouse to enter the dark chamber was recorded, and at this time, the mouse was not given an electric shock. The maximum waiting time was set to 300 seconds, and if the mouse did not enter the dark chamber after the door was opened, the waiting time was recorded as 300 seconds.

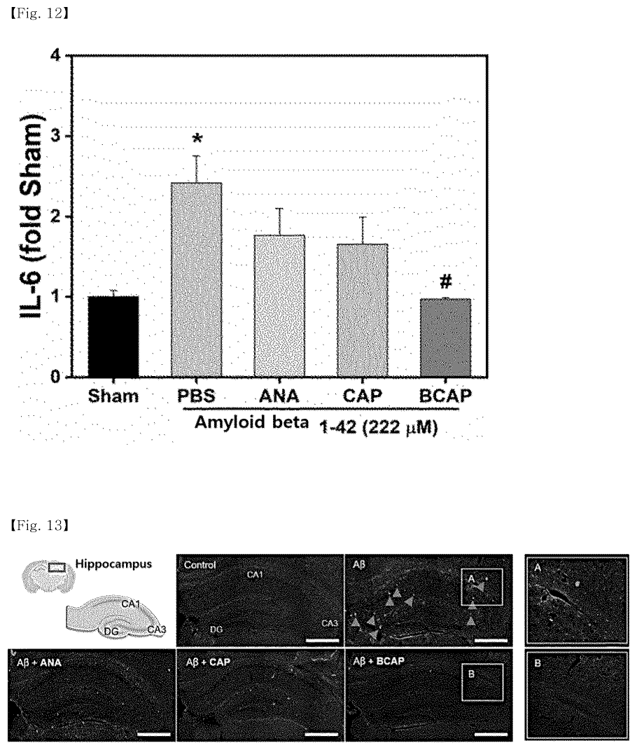

As a result of the passive avoidance test, as shown in FIG. 11, it was confirmed that BCAP could significantly improve memory loss induced by amyloid-beta plaques in the same context as the results of the Y-maze.

These results imply that BCAP has the potential to improve memory loss, one of the main symptoms of Alzheimer's disease.

### 9-4. IL-6 ELISA Analysis

It was well known that amyloid-beta plaques increased the level of interleukin 6 (IL-6). Here, the level of IL-6 in the Alzheimer's disease mouse model according to BCAP treatment was analyzed using enzyme-linked immunosorbent assay (ELISA), one of enzyme-linked immunosorbent assays. Blood collected from the mouse model was centrifuged at 3,000 rpm and 4° C. for 3 minutes, and the plasma (supernatant) was collected for protein analysis. The level of IL-6 in the plasma of the mouse model was measured according to a manufacturer's protocol using an ELISA kit (Novex, Model No. KMC0061, US).

FIG. 12l shows ELISA results for IL-6 changes, in which it was confirmed that the increased level of IL-6 induced by amyloid-beta plaques could also be significantly improved by BCAP.

These immune results were results that proved that BCAP may also improve other symptoms of Alzheimer's disease.

### 9-5. ThS Tissue Staining of Brain Sections

Finally, amyloid-beta plaques in the hippocampus, which was most closely related to Alzheimer's disease and memory among brain tissues, were confirmed by fluorescence imaging using ThS. Specifically, after the passive avoidance test (Example 9-3), mice were sacrificed and perfused with PBS (50 mM, pH 7.4) and fixed (4% paraformaldehyde). After perfusion, the brains were removed by postfixation overnight at 4° C. and cultured in 30% sucrose at 4° C. Coronal sections of 25 μm thickness were sequentially prepared with a cryostat (CM1850; Leica, Wetzlar, Germany) and stored at 4° C. Free-floating sections were incubated in 1% thioflavin S dissolved in 50% ethanol for 10 minutes, then washed twice with 50% ethanol for 5 minutes each and washed once with distilled water for 5 minutes. Then, the sections were stained by applying a mounting medium and fluorescent images were confirmed.

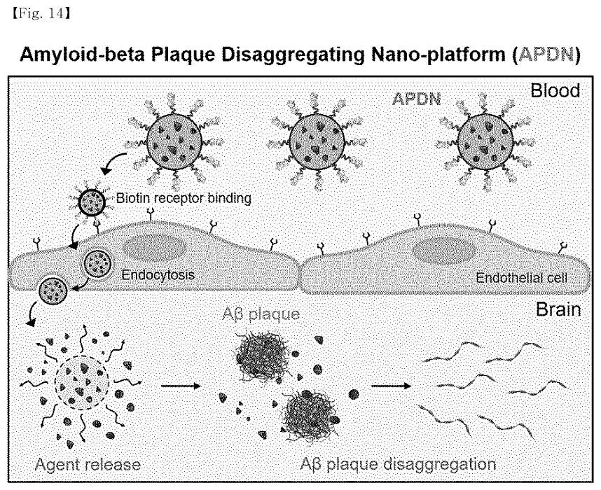

As a result, as shown in FIG. 13, a large amount of amyloid-beta plaques were found in the hippocampus of the Alzheimer's disease mouse model, whereas almost no amyloid-beta plaques were found in the hippocampus of the Alzheimer's disease mouse model administered with BCAP.

As these results, in addition to the previous in vitro results, it was confirmed that BCAP had significant amyloid-beta plaque disaggregation efficacy even in vivo. Therefore, it was clearly confirmed that the nano platform according to the present disclosure may disaggregate the amyloid-beta plaques as shown in the mechanism of action in FIG. 14.

The aforementioned description of the present disclosure is used for exemplification, and it may be understood by those skilled in the art that the present disclosure may be easily modified in other detailed forms without changing the technical spirit or requisite features of the present disclosure. Therefore, it should be appreciated that the aforementioned examples are illustrative in all aspects and are not restricted.

1. A nano platform comprising a compound of Chemical Formula 1, a hydrate thereof, a solvate thereof, or a pharmaceutically acceptable salt thereof:

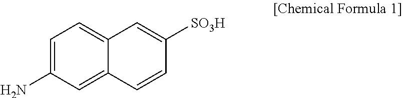 [SMILES: Nc1ccc2cc(S(=O)(=O)O)ccc2c1]

2. The nano platform of claim 1, wherein the compound of Chemical Formula 1, the hydrate thereof, the solvate thereof, or the pharmaceutically acceptable salt thereof is loaded onto porous nanoparticles.

3. The nano platform of claim 2, wherein the porous nanoparticles are porous silicon nanoparticles.

4. The nano platform of claim 1, wherein the nano platform is surface-modified with a brain targeting moiety.

5. The nano platform of claim 4, wherein the brain targeting moiety is biotin-polyethylene glycol (biotin-PEG).

6. The nano platform of claim 2, wherein the porous nanoparticles are porous silicon nanoparticles of which the surface is sealed with calcium chloride and functionalized with biotin-polyethylene glycol (biotin-PEG).

7. The nano platform of claim 1, wherein the nano platform exhibits targeting activity to the brain in vivo.

8. The nano platform of claim 1, wherein the nano platform has amyloid-beta plaque disaggregation efficacy.

9. A manufacturing method of the nano platform of claim 1, comprising:
preparing porous silicon nanoparticles;
loading a compound of the following Chemical Formula 1, a hydrate thereof, a solvate thereof, or a pharmaceutically acceptable salt thereof onto the porous silicon nanoparticles;
sealing the surface of the loaded porous silicon nanoparticles with calcium chloride; and
surface-modifying the coated porous silicon nanoparticles with biotin-polyethyleneglycerol (biotin-PEG):

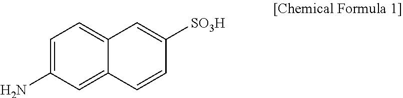 [SMILES: Nc1ccc2cc(S(=O)(=O)O)ccc2c1]

10. The manufacturing method of claim 9, wherein the sealing with calcium chloride is performed by reacting the porous silicon nanoparticles with calcium or magnesium.

11. A method for preventing or treating a disease caused by amyloid-beta plaques, comprising administering to a subject in need thereof the nano platform of claim 1.

12. The method of claim 11, wherein the disease caused by the amyloid-beta plaques is at least one selected from the group consisting of Alzheimer's disease, Parkinson's disease, stroke, Down syndrome, amyloid angiopathy, systemic amyloidosis, Dutch amyloidosis, inclusion body myositis, Creutzfeldt-Jakob disease, Kennedy's disease, Amyotrophic Lateral Sclerosis, Fronto-Temporal Dementia, Cortico-Basal Degeneration, Huntington's disease, senile dementia of the Alzheimer type, Lewis body dementia, vascular dementia, mild cognitive impairment, and age-related memory impairment.
